(19) United States
(12) Patent Application Publication
(10) Pub. No.: US 2024/0139435 A1
(43) Pub. Date: May 2, 2024

(54) VASCULAR PUNCTURE ASSISTANCE DEVICE

(71) Applicant: TERUMO KABUSHIKI KAISHA, Tokyo (JP)

(72) Inventors: Takito INUKAI, Isehara-shi (JP); Yoichiro KUWANO, Tokyo (JP); Takumi FUKUDA, Hadano-shi (JP)

(73) Assignee: TERUMO KABUSHIKI KAISHA, Tokyo (JP)

(21) Appl. No.: 18/408,695

(22) Filed: Jan. 10, 2024

Related U.S. Application Data
(63) Continuation of application No. PCT/JP2022/026824, filed on Jul. 6, 2022.

(30) Foreign Application Priority Data
Jul. 21, 2021 (JP) 2021-120217

(51) Int. Cl. A61M 5/42

(52) U.S. Cl. CPC A61M 5/427; A61M 2205/3303; A61M 2210/12

(57) ABSTRACT

A vascular puncture assist device includes a property acquisition unit that acquires a property of a blood vessel; and a control unit that detects a state of hardness of a blood vessel from the property of the blood vessel acquired by the property acquisition unit, and determines availability of puncture and a puncture position or direction based on the detected state of hardness of the blood vessel. The vascular puncture assist device further includes a probe including an imaging unit that acquires a cross-sectional image of a human body, and the control unit detects a state of calcification of a blood vessel from the acquired cross-sectional image using the imaging unit as a property acquisition unit.

### CROSS-REFERENCES TO RELATED APPLICATIONS

This application is a continuation of International Application No. PCT/JP2022/026824 filed on Jul. 6, 2022, which claims priority to Japanese Application No. 2021-120217 filed on Jul. 21, 2021, the entire content of both of which is incorporated herein by reference.

### TECHNOLOGICAL FIELD

The present disclosure generally relates to a vascular puncture assist device that detects a property of a blood vessel to be punctured.

### BACKGROUND DISCUSSION

In order to secure an access site for drug administration and endovascular treatment, vascular puncture for puncturing a human body with an injection needle is performed. In the vascular puncture, an operator cannot visually observe the blood vessel from a skin surface, and thus, a position of the blood vessel is estimated by standard knowledge of blood vessel running and skill such as tactile perception of blood vessel pulsation. However, a vascular puncture failure often occurs, which causes physical and mental distress to a patient.

In order to specify a puncture position, in recent years, a technique for visualizing a position of a blood vessel through a near-infrared image, an ultrasound echo, photoacoustic imaging, or the like, is used. By visualizing the blood vessel position, the operator can relatively easily determine the puncture position, the puncture angle, the puncture depth, or the like. In addition, a device that drives a needle based on obtained information of a blood vessel position and automatically performs puncture is also known. As a device for automatically performing puncture, for example, there is a device described in U.S. Pat. No. 9,364,171.

The blood vessel to be punctured may be partially calcified. Calcification often occurs in the intima and media of blood vessels. Since the calcified site of the blood vessel is hardened, puncture may fail depending on the place and angle of puncture. Therefore, it is required to enable puncture avoiding a calcified location or puncture at an angle that is hardly affected by calcification.

### SUMMARY

A vascular puncture assist device is disclosed that detects the property of a blood vessel before puncture and assists reliable puncture.

A vascular puncture assist device according to the present disclosure includes a control unit that determines information necessary for puncture based on information from a property acquisition unit that is configured to acquire a property of a blood vessel, in which the control unit is configured to detect a state of hardness of a blood vessel from the property of the blood vessel acquired by the property acquisition unit, and determine availability of puncture and a puncture position or direction based on the detected state of hardness of the blood vessel.

A vascular puncture assist device is disclosed comprising: a control unit configured to acquire a property of a blood vessel, detect a state of hardness of a blood vessel from the property of the blood vessel, and determine availability of puncture of the blood vessel and a puncture position or direction based on the detected state of hardness of the blood vessel.

A method is disclosed for puncturing a blood vessel, the method comprising: acquiring a property of the blood vessel; detecting a state of hardness of the blood vessel from the acquired property of the blood vessel; and determining availability of the puncturing of the blood vessel and a puncture position or direction based on the detected state of hardness of the blood vessel.

In the vascular puncture assist device configured as described above, the availability of puncture and the position and angle of puncture can be determined according to the condition of the hardness of the blood vessel, in a manner that the possibility of puncture failure due to the influence of a site where the blood vessel is hardened, such as a calcified tissue, can be reduced.

### BRIEF DESCRIPTION OF THE DRAWINGS

FIG. 1 is a front view of a vascular puncture device including a vascular puncture assist device according to the present embodiment.

FIG. 2 is a perspective view illustrating an internal structure of a needle drive unit.

FIGS. 3A and 3B are plan views illustrating a positional relationship between a blood vessel and a needle, in which FIG. 3A is a view illustrating a case where an orientation of the needle with respect to the blood vessel is changed, and FIG. 3B is a view illustrating a case where a position of the needle with respect to the blood vessel is changed.

FIG. 4 is a cross-sectional view illustrating a positional relationship between a blood vessel and a needle, and is a view illustrating a case where an incident angle of the needle with respect to the blood vessel is changed.

FIG. 5 is a view illustrating a skin contact surface of a probe and is a view illustrating a positional relationship with an arm for which a cross-sectional image is to be acquired.

FIG. 6 is a configuration diagram of the vascular puncture assist device.

FIG. 7 is a flowchart of puncture using the vascular puncture device.

FIG. 8 is a conceptual diagram of an acquired echogram.

FIGS. 9A to 9D are cross-sectional views of a blood vessel having a calcified portion.

FIG. 10 is a view illustrating a positional relationship between a position of center of gravity, imaging position, and puncture point of a blood vessel.

FIG. 11 is a side view of a probe including a notification unit.

FIG. 12 is a flowchart of puncture after puncture timing determination in a case where the notification unit makes notification of puncture timing.

FIG. 13 is a flowchart after puncture is executed in S6 of FIG. 7.

FIG. 14 is a schematic view of a vascular puncture assist device capable of acquiring a cross-sectional image and automatically performing puncture.

### DETAILED DESCRIPTION

Set forth below with reference to the accompanying drawings is a detailed description of embodiments of a vascular puncture assist device that detects a property of a blood vessel to be punctured. Note that dimensional ratios of the drawings are exaggerated for the convenience of description and may differ from actual ratios in some cases.

A vascular puncture assist device 10 according to the embodiment of the present disclosure can be used when puncturing an arm of a human body. The vascular puncture assist device 10 acquires a cross-sectional image of the arm, detects a blood vessel position, determines a puncture timing and puncture parameters, and punctures the blood vessel with a needle.

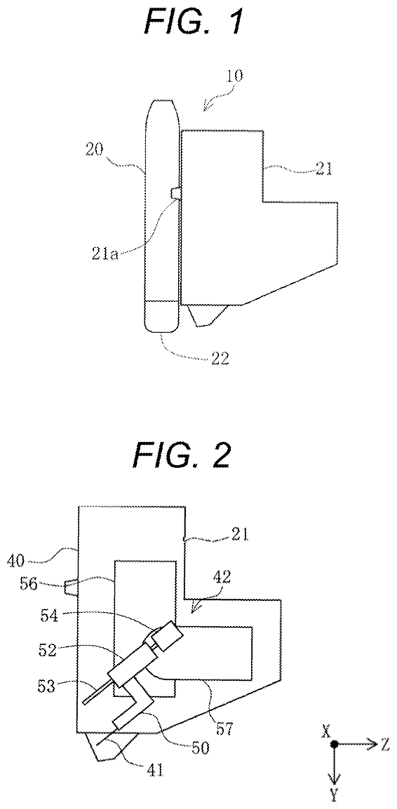

As illustrated in FIG. 1, a vascular puncture assist device 10 can include a probe 20 including an imaging unit 22 that comes into contact with the skin surface to acquire the cross-sectional image of the human body, and a puncture unit 21 that performs puncture by driving a needle. The puncture unit 21 has a fixing portion 21a engaged with and fixed to the probe 20, and the probe 20 and the puncture unit 21 are integrated.

As illustrated in FIG. 2, the puncture unit 21 includes a body portion 40, a needle 41 for puncture, and a drive unit 42 for moving the needle 41. The drive unit 42 includes a horizontal drive unit 56 assisted by the body portion 40, a biaxial drive unit 57 assisted by the horizontal drive unit 56, a puncture direction drive unit 54 assisted by the biaxial drive unit 57, a puncture direction guide unit 53 assisted by the puncture direction drive unit 54, and a puncture movement unit 52 assisted by the puncture direction guide unit 53 and movable along the puncture direction. FIG. 2 illustrates an X-Y-Z direction. The X direction corresponds to the width direction of the arm, the Y direction corresponds to the depth direction of the arm, and the Z direction corresponds to the length direction of the arm. The needle 41 is fixed to a needle holding portion 50 provided in the puncture movement unit 52.

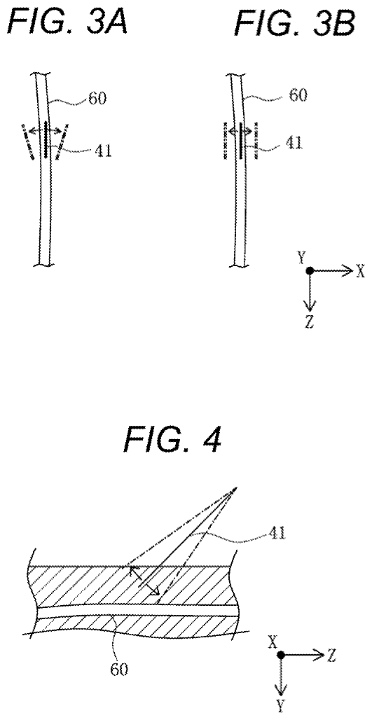

The biaxial drive unit 57 allows the needle 41 to change its orientation with respect to the X-Z flat surface. As a result, as illustrated in FIG. 3A, it is possible to cause the needle 41 to enter a blood vessel 60 straight by changing the orientation of the needle 41 with respect to the flat surface direction of the skin surface. The horizontal drive unit 56 allows the needle 41 to move in the X direction. As a result, as illustrated in FIG. 3B, the needle 41 can be moved along the flat surface direction of the skin surface and disposed in a manner that the needle 41 can be incident on the blood vessel 60. The biaxial drive unit 57 allows the needle 41 to change its inclination in the Y-Z flat surface. As a result, as illustrated in FIG. 4, the incident angle of the needle 41 with respect to the skin surface can be changed.

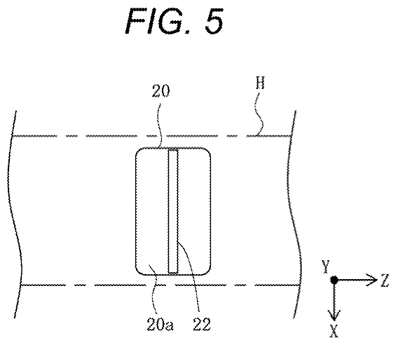

As illustrated in FIG. 5, the imaging unit 22 of the probe 20 is provided to extend along one direction at a central portion of a skin contact surface 20a of the probe 20 and extend over substantially the entire width of the probe 20. The imaging unit 22 can be an echographic apparatus that includes a transducer that generates an ultrasound wave and obtains a cross-sectional image of the inside of the human body by detecting the reflected wave. In the present embodiment, the cross-sectional image orthogonal to an axial direction of the blood vessel is acquired, and thus, the imaging unit 22 is disposed such that a length direction of the imaging unit 22 is orthogonal to a length direction of an arm H. In addition, in a case where the blood vessel runs largely meandering, the imaging unit 22 is disposed obliquely with respect to the length direction of the arm H.

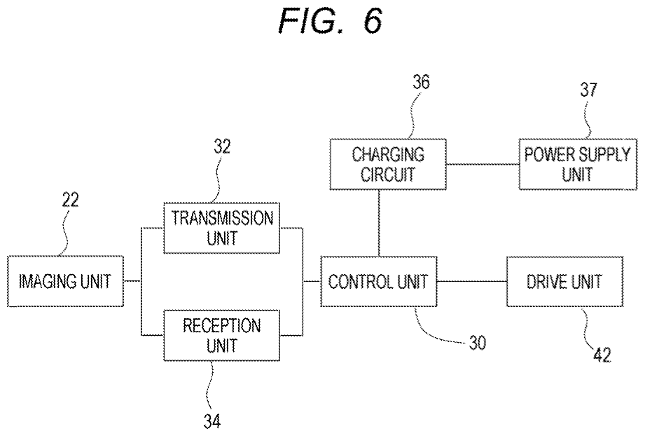

As illustrated in FIG. 6, the vascular puncture assist device 10 includes the imaging unit 22 that comes into contact with a skin surface to acquire a cross-sectional image of a human body, and a control unit 30 that detects a blood vessel position from the cross-sectional image. The control unit 30 is connected to the imaging unit 22 via a transmission unit 32 and a reception unit 34 and can cause the imaging unit 22 to acquire a cross-sectional image and receive the acquired cross-sectional image. In addition, the control unit 30 can control the drive unit 42 to perform puncture with the needle 41. The control unit 30 can be connected to a power supply unit 37 including a rechargeable battery via a charging circuit 36.

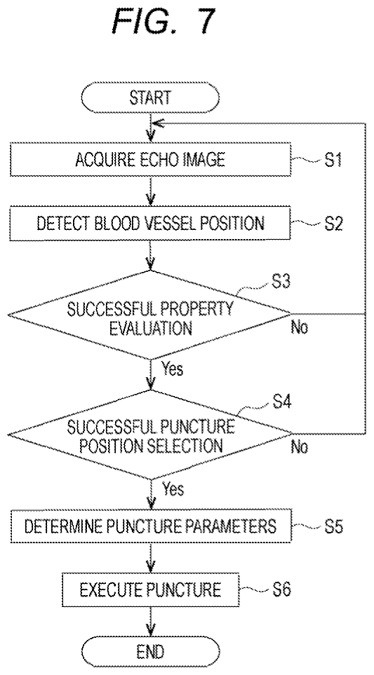

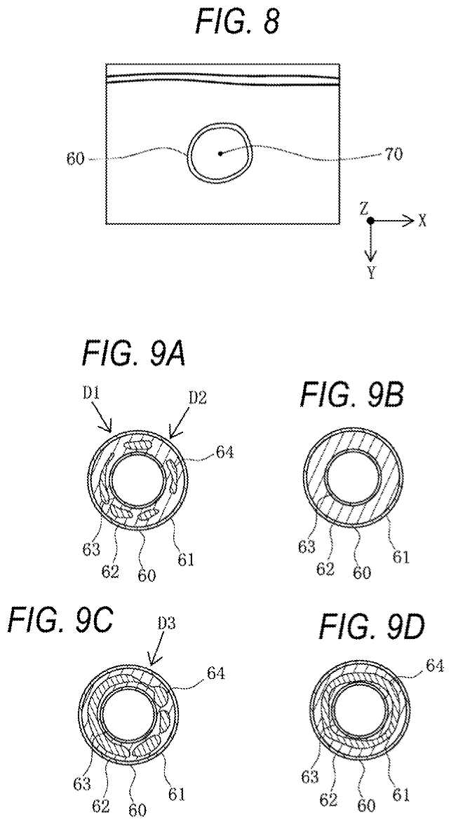

Next, the operation of the vascular puncture assist device 10 will be described along the flow of puncture. As illustrated in FIG. 7, the control unit 30 acquires a cross-sectional image as illustrated in FIG. 8 from the imaging unit 22 (S1). The horizontal direction in the cross-sectional image is the X direction, the vertical direction in the cross-sectional image is the Y direction, and the direction orthogonal to the paper surface of the cross-sectional image is the Z direction. Coordinates of an upper left point in the cross-sectional image are set as a start point (0, 0, 0). The position and property of the blood vessel and the property around the blood vessel can be detected from the cross-sectional image acquired by the imaging unit 22. That is, the imaging unit 22 functions as a property acquisition unit that acquires a property of a blood vessel. The property acquisition unit may be other than the imaging unit 22. For example, a CT device or the like can be used as the property acquisition unit. Examples of the property of the blood vessel include the inner diameter of the blood vessel, the flatness of the blood vessel, the thickness of the vessel wall, the presence or absence, the position, and the thickness of fibrous plaque in the blood vessel or the membrane, and the presence or absence, the position, and the thickness of calcified tissue in the blood vessel or the membrane. In addition, examples of the property around the blood vessel include a distance from the skin to the blood vessel, a tissue state between the skin and the blood vessel (for example, thickening due to fibrous tissue, and presence or absence of connective tissue such as tendon), and a tissue state behind or on the side of the puncture direction of the blood vessel (for example, distance to radius, and presence or absence of connective tissue such as a tendon).

The control unit 30 detects the position of the blood vessel 60 in the image by analyzing the acquired cross-sectional image (S2). The control unit 30 detects a region recognized as the blood vessel 60 in the image, and sets a position 70 of the center of gravity as the position of the blood vessel. In order to detect the region recognized as the blood vessel in the image, it is possible to prepare a large number of images of the same type and use a machine learning or deep learning method. In addition, it is also possible to detect a region with blood flow by the Doppler method in the imaging unit 22 and recognize the region as the region of the blood vessel. When the region of the blood vessel is detected from the cross-sectional image, it is necessary to perform detection while distinguishing the artery from the vein. The artery can be distinguished from the vein on the basis of a position of the bone of the arm H appearing in the cross-sectional image. In addition, when the region with the blood flow is detected by the Doppler method, the artery and the vein can be distinguished by a direction of the blood flow. Coordinates of the detected position 70 of the center of gravity of the blood vessel are defined as (x, y, 0).

The control unit 30 evaluates the property of the blood vessel from the cross-sectional image acquired by the imaging unit 22 (S3). The property of the blood vessel evaluated in this example is the presence or absence, the position, and the thickness of calcified tissue in the blood vessel or membrane. The state of the hardness of the blood vessel can be detected by evaluating the state of the calcified tissue. In a case where the property of the blood vessel can be evaluated in S3, the process proceeds to S4. In a case where the property of the blood vessel cannot be evaluated, the process is repeated again from the acquisition of the echogram (S1).

In the evaluation of the property of the blood vessel, the range of the calcified site can be estimated using a machine learning or deep learning method from the difference in brightness value in the cross-sectional image acquired by the imaging unit 22. In addition, in the evaluation of the property of the blood vessel, the range of the calcified site may be estimated using a machine learning or deep learning method from the acoustic shadow in the cross-sectional image acquired by the imaging unit 22. In addition, since the calcified site is relatively hard, the range of the calcified site may be estimated using elastography.

When the property of the blood vessel can be evaluated, the control unit 30 selects a puncture position (S4). In the selection of the puncture position, availability of puncture and the position or direction of puncture are determined based on the property evaluation of the blood vessel. As illustrated in FIG. 9A, the blood vessel 60 has a media 62 having high elasticity between a thin adventitia 61 and an intima 63. In the membrane of the blood vessel 60, a calcified tissue 64 exists in a part in the circumferential direction. In this case, the control unit 30 determines that puncture is possible and determines a direction D1 or a direction D2 in which the calcified tissue 64 does not exist as the puncture direction.

In a case where the probe 20 is moved in the length direction of the blood vessel 60 and there is a position where the calcified tissue 64 does not exist as illustrated in FIG. 9B, the control unit 30 determines the position as the puncture position.

As illustrated in FIG. 9C, in a case where the calcified tissue 64 exists over substantially the entire circumference and the thickness of a part of the calcified tissue in the circumferential direction is small, the control unit 30 determines a direction D3 in which the thickness of the calcified tissue 64 is relatively small as the puncture direction.

As illustrated in FIG. 9D, in a case where the calcified tissue 64 exists over the entire circumference and the thickness is also large over the entire circumference, the control unit 30 determines that the position is not puncturable.

Note that, in FIGS. 9A to 9D, the calcified tissue 64 exists in the media 62, but the same applies to a case where the calcified tissue 64 exists in the adventitia 61 or the intima 63.

When the puncture is possible and the position or direction of the puncture can be determined in S4, the process proceeds to S5. In a case where it is determined that the puncture is impossible in S4 or in a case where the position or direction of the puncture cannot be determined, the position of the probe 20 is changed to the blood vessel direction, and the process is repeated again from the acquisition of the echogram. In addition, in a case where the probe 20 is connected to the probe drive unit and it is determined that puncture is impossible, the probe drive unit can automatically change the position of the probe 20 in the direction of the blood vessel and specify the position of the blood vessel in which no calcified tissue exists in the blood vessel based on the acquired echogram.

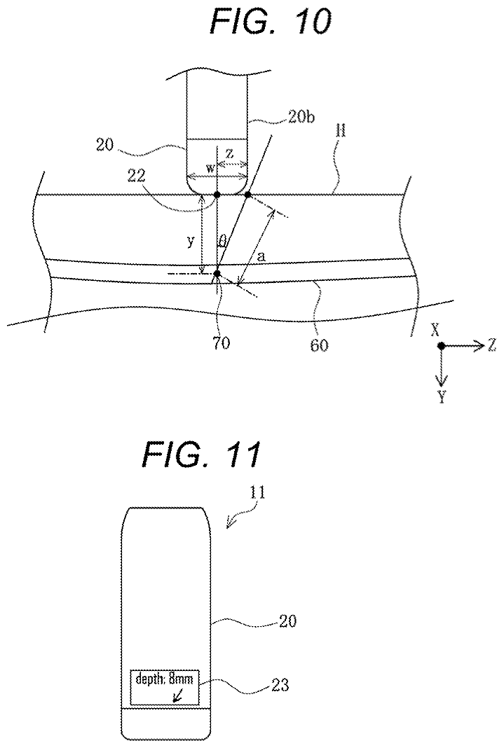

When the position or direction of puncture is determined in S4, the control unit 30 determines parameters necessary for puncture (S5). Parameters required for puncture include a puncture direction on the X-Z flat surface, a puncture position, an incident angle of the needle 41, a puncture depth of the needle 41, and a puncture speed of the needle 41. In a case where the puncture direction is determined in S4, the puncture direction on the X-Z flat surface is set to that direction. In a case where the puncture direction is not specified in S4, as illustrated in FIG. 10, since the position 70 of the center of gravity of the blood vessel is detected from the cross-sectional image acquired by the imaging unit 22, the puncture direction in the X-Z flat surface is determined in a manner that the needle 41 of the puncture unit 21 fixed to the probe 20 faces the position 70 of the center of gravity.

The puncture position is a position immediately below a side surface 20b of the probe 20. In this case, a coordinate z in the Z direction of the side surface 20b of the probe 20 to be the puncture position is half a width W of the probe 20, and thus, is calculated by z=W/2. The puncture angle is an angle θ of a line from the position of the center of gravity of the blood vessel to the puncture position relative to a perpendicular line of the skin surface, and is calculated by θ=arctan(z/y). The puncture depth a is calculated by a=y/cos θ. As a result, the coordinate of the puncture position in the x direction and a puncture depth a of the needle 41 are defined.

As the parameter of puncture, other parameters may be used. For example, puncture start time, puncture completion time, puncture acceleration at the time of puncture start, puncture acceleration at the time of puncture completion, or the like can be used.

In addition, in determining the parameters of puncture, the properties of the blood vessel, specifically, the inner diameter of the blood vessel, the flatness of the blood vessel, the thickness of the vessel wall, the presence or absence, the position, and the thickness of fibrous plaque in the blood vessel or the membrane may be considered. For example, from these properties, the hardness of the tissue of the blood vessel is evaluated, and the harder the tissue, the closer the puncture angle is to vertical. Thus, reliable puncture can be executed according to the hardness of the tissue. In addition, from these properties, the hardness of the tissue of the blood vessel is evaluated, and the harder the tissue, the faster the puncture speed is. Thus, reliable puncture can be executed according to the hardness of the tissue. In addition, in determining the parameters of puncture, the property around the blood vessel, specifically, a distance from the skin to the blood vessel, a tissue state between the skin and the blood vessel (for example, thickening due to fibrous tissue, and presence or absence of connective tissue such as tendon), and a tissue state behind or on the side of the puncture direction of the blood vessel (for example, distance to radius, and presence or absence of connective tissue such as tendon) can be considered. For example, from these properties, the hardness of the tissue of the blood vessel from the surface of the skin to the inside of the blood vessel is evaluated, and the harder the tissue, the closer the puncture angle is to vertical. Thus, reliable puncture can be executed according to the hardness of the tissue. In addition, from these properties, the hardness of the tissue of the blood vessel from the surface of the skin to the inside of the blood vessel is evaluated, and the harder the tissue, the faster the puncture speed is. Thus, reliable puncture can be executed according to the hardness of the tissue.

After determining the parameters of puncture, the control unit 30 operates the drive unit 42 according to the parameters of puncture to execute puncture (S6).

Next, a modification of the vascular puncture assist device will be described. The vascular puncture assist device 11 can also be used in a case where the operator manually performs puncture. As illustrated in FIG. 11, the vascular puncture assist device 11 includes only the probe 20, and a display unit 23 that displays parameters of puncture is provided on a side surface of the probe 20.

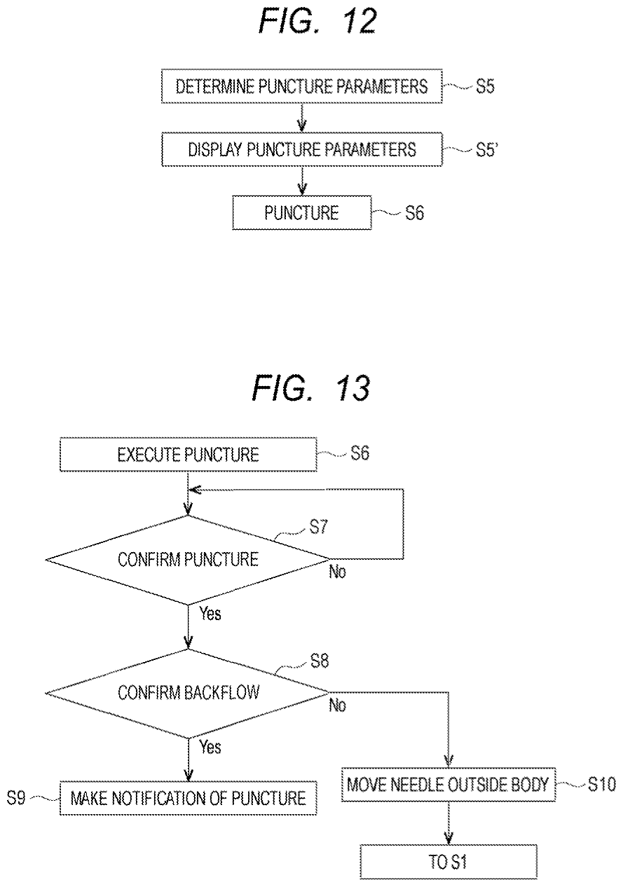

The operation at the time of puncture is the same in the present example from S1 to S5 in FIG. 7. As illustrated in FIG. 12, after determining the parameters of puncture, the control unit 30 displays the parameters of puncture on the display unit 23 (S5′). Parameters of puncture displayed on the display unit 23 are, for example, a puncture depth and a puncture direction. As illustrated in FIG. 11, the puncture depth can be displayed in letters. In addition, the direction of puncture can be indicated by an arrow. The operator can perform the puncture while viewing the parameters of the puncture displayed on the display unit 23 (S6). Regarding the direction of puncture, puncture can be performed in the determined direction by puncturing the needle along the arrow.

After executing the puncture, the vascular puncture assist device 10 may operate as follows. In this case, the puncture unit 21 includes an optical sensor capable of detecting the backflow of blood at the proximal portion of the needle 41. As illustrated in FIG. 13, when the puncture is executed in S6, the control unit 30 determines whether or not the needle 41 has reached the blood vessel 60 from the cross-sectional image acquired by the imaging unit 22 (S7). When the needle 41 reaches the blood vessel 60, the control unit 30 detects the presence or absence of backflow of blood by the optical sensor (S8). When the backflow of blood is detected, the control unit 30 causes a notification unit to make notification that puncture has been performed (S9). In addition, the control unit 30 may proceed to a step executed after puncture instead of the notification. In a case where the backflow of blood is not confirmed in S8, the control unit 30 moves the needle 41 to the outside of the body by the drive unit 42 (S10), returns to S1, acquires the cross-sectional image, and detects the blood vessel position again.

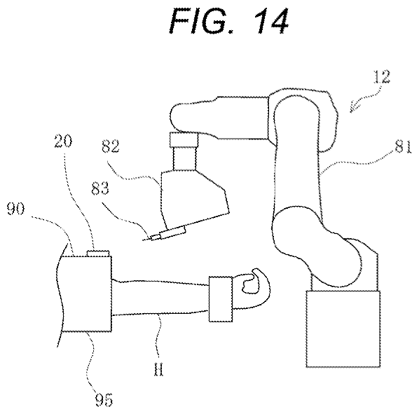

In addition, the vascular puncture assist device 12 may be an automatic puncture device capable of automatically performing the puncture of the arm H including acquisition of a cross-sectional image. As illustrated in FIG. 14, the vascular puncture assist device 12 includes a robot arm 81 capable of three-dimensionally moving a distal portion 82 to which a needle 83 is attached, and the probe 20. The robot arm 81 can perform puncture with the needle 83 from an arbitrary position at an arbitrary angle by control based on a sensor.

If the arm H is inserted into the base portion 90 and fixed by the fixing portion 95, a cross-sectional image is acquired by the probe 20, and the control unit 30 determines the puncture timing and the puncture parameters from the cross-sectional image. The robot arm 81 punctures the arm H with the needle 83 according to the determined availability of puncture, puncture position or direction, and puncture parameters.

At the time of puncture, in a case where the needle 83 is not detected in the cross-sectional image even after a predetermined constant time has elapsed from the start of puncture, it is determined that the needle 83 has not reached the blood vessel and the puncture has failed. In a case where the puncture fails, a notification is made, and the needle 83 is returned to the original position by the robot arm 81.

As described above, the vascular puncture assist device 10 according to the present embodiment a vascular puncture assist device 10 including a control unit 30 that determines information necessary for puncture based on information from a property acquisition unit that acquires a property of a blood vessel, in which the control unit 30 detects a state of calcification of a blood vessel from the property of the blood vessel acquired by the property acquisition unit, and determines availability of puncture and a puncture position or direction based on the detected state of calcification of the blood vessel. In the vascular puncture assist device 10 configured as described above, the availability of puncture and the position and angle of puncture can be determined according to the condition of the calcification of the blood vessel, in a manner that the possibility of puncture failure due to the influence of a calcified tissue, can be reduced.

In addition, a probe 20 including an imaging unit 22 that acquires a cross-sectional image of a human body may further be included, and the control unit 30 may detect a state of calcification of a blood vessel from the acquired cross-sectional image using the imaging unit 22 as a property acquisition unit. As a result, the property of the blood vessel can be rather easily acquired from the cross-sectional image.

In addition, a puncture unit including a needle that punctures a blood vessel, and a drive unit that moves the needle to execute puncture may further be included, and the control unit 30 may cause the puncture unit to execute puncture at the determined puncture position or direction. As a result, since the needle 41 is automatically punctured by the puncture unit 21, puncture can be reliably executed at the puncture position and angle determined by the control unit 30.

In addition, the probe 20 may include a notification unit that makes notification of a position or a direction of puncture, and the control unit 30 may cause the notification unit to make notification of the determined position or direction of the puncture. As a result, in a case where the puncture is manually performed, the operator can rather easily grasp the position and angle of the puncture.

In addition, the property acquisition unit may acquire a property of the blood vessel and a property around the blood vessel, and the control unit 30 may determine a parameter necessary for executing puncture based on the property of the blood vessel and the property around the blood vessel acquired by the property acquisition unit. As a result, the reliability of puncture can be further improved.

In addition, the control unit 30 may detect a state of calcification of the blood vessel as the state of the blood vessel. As a result, the hardness of the blood vessel can be appropriately evaluated.

In addition, the control unit 30 may detect a state of fibrous plaque of the blood vessel as the state of the blood vessel. As a result, the hardness of the blood vessel can be appropriately evaluated.

The present disclosure is not limited to the above-described embodiment, and various modifications can be made by those skilled in the art within the technical idea of the present invention. For example, although a monitor that displays the cross-sectional images acquired in the present embodiment is not illustrated, the vascular puncture assist device 10 may be connected to the monitor such that the cross-sectional images can be visually observed.

In addition, in the present embodiment, the target position of puncture is set as the position of the center of gravity of the blood vessel to be punctured, but a position other than the position of center of gravity of the blood vessel to be punctured may be set as the target position. For example, an inner surface of a blood vessel positioned between a blood vessel to be punctured and the imaging unit 22 or a position in a membrane of the blood vessel may be detected, and the position may be set as the target position. In addition, a position separated from these positions by a certain distance may be set as the target position.

In S3 of FIG. 7, the property of the blood vessel evaluated by the control unit 30 may be the state of the fibrous plaque of the blood vessel. Since the site of the fibrous plaque is displayed with higher brightness than the normal blood vessel in the cross-sectional image acquired by the imaging unit 22, the control unit 30 can determine the site. In addition, the calcified site has high brightness and generates an acoustic shadow in the cross-sectional image acquired by the imaging unit 22, whereas the site of the fibrous plaque has high brightness and does not generate an acoustic shadow in the cross-sectional image acquired by the imaging unit 22. Therefore, the control unit 30 can determine these sites.

In addition, a color map of the hardness of the tissue can be created by elastography. Based on this, the control unit 30 can also determine the hardness of the blood vessel. Furthermore, the hardness of the blood vessel can be evaluated from the cross-sectional image acquired by the imaging unit 22. These techniques may be combined in the evaluation of the property of the blood vessel in S3. By discriminating (or determining) and evaluating a plurality of properties of the blood vessel, the control unit 30 can reflect the properties in a position and a direction suitable for puncture and other puncture parameters, and can execute puncture based on these. This makes it possible to further reduce the risk of failing puncture.

In addition, the vascular puncture assist device 10 may have a function of displaying a blood vessel determined to be punctured or a medical device adapted to the punctured blood vessel. In a case where the puncture unit includes a hollow inner needle and a flexible tubular outer tube disposed to cover the outer peripheral surface of the inner needle, the operator inserts, for example, a sheath along the outer tube after puncturing the blood vessel with the puncture unit and removing the inner needle. The outer diameter of the sheath is preferably equal to or smaller than the inner diameter of the blood vessel to be inserted. This is because in a case where the outer diameter of the sheath is equal to or larger than the inner diameter of the blood vessel, a complication is likely to occur by inserting the sheath into the blood vessel. As an example of a method of calculating the blood vessel inner diameter, the length of the diagonal line passing through the center of gravity of the inner peripheral surface of the specified blood vessel (artery or vein) can be acquired for the entire circumference at predetermined angle intervals (for example, in increments of 1 degree), and the average value can be set as the blood vessel inner diameter. In addition, the inner diameter of the blood vessel may be calculated back from the inner area of the inner peripheral surface of the blood vessel. Since the artery pulsates, it is preferable to detect the blood vessel inner diameter at a certain timing when detecting the blood vessel inner diameter of the artery. In addition, the certain timing is preferably when the blood vessel is most contracted. Since the minimum inner diameter of the blood vessel inner diameter is larger than the outer diameter of the medical device to be inserted, the occurrence of complications can be reduced. After calculating the blood vessel inner diameter, the control unit 30 can display the outer diameter and the type of the mobile device suitable for the calculated blood vessel inner diameter on a display device (or display unit 23) such as a monitor together with the cross-sectional image. The control unit 30 may specify at least one of the optimal outer diameter, length, or variety of the inner needle from information on the blood vessel determined to be punctured, past statistical information, or the like, display the same on a display device such as a monitor together with a cross-sectional image, and present the same to the operator.

In addition, the vascular puncture assist device 10 includes the control unit 30 that determines information necessary for puncture based on information from the property acquisition unit, and the property acquisition unit may be provided separately from the vascular puncture assist device 10, or the property acquisition unit may be integrally configured with the vascular puncture assist device 10 as described above.

The detailed description above describes embodiments of a vascular puncture assist device that detects a property of a blood vessel to be punctured. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A vascular puncture assist device comprising:
a control unit configured to determine information for puncture based on information from a property acquisition unit configured to acquire a property of a blood vessel; and
wherein the control unit is configured to detect a state of hardness of a blood vessel from the property of the blood vessel acquired by the property acquisition unit, and determine availability of puncture and a puncture position or direction based on the detected state of hardness of the blood vessel.

2. The vascular puncture assist device according to claim 1, further comprising:
a probe including an imaging unit configured to acquire a cross-sectional image of a human body; and
wherein the control unit is configured to use the imaging unit as the property acquisition unit and detect the state of hardness of the blood vessel from the acquired cross-sectional image of the human body.

3. The vascular puncture assist device according to claim 1, further comprising:
a puncture unit, the puncture unit including a needle configured to puncture a blood vessel, and a drive unit configured to move the needle to execute the puncture of the blood vessel; and
wherein the control unit is configured to cause the puncture unit to execute puncture of the blood vessel at the determined puncture position or direction.

4. The vascular puncture assist device according to claim 1, wherein
the probe includes a notification unit configured to provide notification of puncture position or direction; and
the control unit is configured to cause the notification unit to make notification of the determined puncture position or direction.

5. The vascular puncture assist device according to claim 1, wherein
the property acquisition unit is configured to acquire a property of the blood vessel and a property around the blood vessel; and
the control unit is configured to determine a parameter necessary for executing puncture based on the property of the blood vessel and the property around the blood vessel acquired by the property acquisition unit.

6. The vascular puncture assist device according to claim 1, wherein the control unit is configured to detect a state of calcification of the blood vessel as a state of the blood vessel.

7. The vascular puncture assist device according to claim 1, wherein the control unit is configured to detect a state of fibrous plaque of the blood vessel as a state of the blood vessel.

8. A vascular puncture assist device comprising:
a control unit configured to acquire a property of a blood vessel, detect a state of hardness of a blood vessel from the property of the blood vessel, and determine availability of puncture of the blood vessel and a puncture position or direction based on the detected state of hardness of the blood vessel.

9. The vascular puncture assist device according to claim 8, further comprising:
an imaging unit configured to acquire a cross-sectional image of a human body; and
wherein the control unit is configured to detect the state of hardness of the blood vessel from the acquired cross-sectional image of the human body.

10. The vascular puncture assist device according to claim 8, further comprising:
a puncture unit, the puncture unit including a needle configured to puncture the blood vessel, and a drive unit configured to move the needle to execute the puncture of the blood vessel; and
wherein the control unit is configured to cause the puncture unit to execute puncture of the blood vessel at the determined puncture position or direction of the blood vessel.

11. The vascular puncture assist device according to claim 8, further comprising:
a notification unit configured to provide notification of puncture position or direction of the blood vessel; and
the control unit is configured to cause the notification unit to make notification of the determined puncture position or direction of the blood vessel.

12. The vascular puncture assist device according to claim 8, wherein the control unit is configured to determine a parameter necessary for executing puncture based on the acquired property of the blood vessel and an acquired property around the blood vessel.

13. The vascular puncture assist device according to claim 8, wherein the control unit is configured to detect a state of calcification of the blood vessel as a state of the blood vessel.

14. The vascular puncture assist device according to claim 8, wherein the control unit is configured to detect a state of fibrous plaque of the blood vessel as a state of the blood vessel.

15. A method for puncturing a blood vessel, the method comprising:
acquiring a property of the blood vessel;
detecting a state of hardness of the blood vessel from the acquired property of the blood vessel; and
determining availability of the puncturing of the blood vessel and a puncture position or direction based on the detected state of hardness of the blood vessel.

16. The method according to claim 15, further comprising:
acquiring a cross-sectional image of a human body; and
detecting the state of hardness of the blood vessel from the acquired cross-sectional image of the human body.

17. The method according to claim 15, further comprising:
executing a puncture of the blood vessel at the determined puncture position or direction.

18. The method according to claim 15, further comprising:
providing a notification of the puncture position or direction to a display device.

19. The method according to claim 15, further comprising:
acquiring a property of the blood vessel and a property around the blood vessel; and
determining a parameter necessary for executing puncture based on the acquired property of the blood vessel and the acquired property around the blood vessel.

20. The method according to claim 15, further comprising:
detecting a state of calcification of the blood vessel as a state of the blood vessel, or
detecting a state of fibrous plaque of the blood vessel as a state of the blood vessel.
